(19) United States
(12) Patent Application Publication
(10) Pub. No.: US 2018/0256642 A1
(43) Pub. Date: Sep. 13, 2018

(54) BIOMATERIALS DERIVED FROM TISSUE EXTRACELLULAR MATRIX

(71) Applicant: The Trustees of Columbia University in the City of New York, New York, NY (US)

(72) Inventors: Donald O. Freytes, Summit, NJ (US); Gordana Vunjak-Novakovic, New York, NY (US); John O'Neill, New York, NY (US)

(21) Appl. No.: 15/913,237

(22) Filed: Mar. 6, 2018

Related U.S. Application Data
(63) Continuation of application No. 14/450,020, filed on Aug. 1, 2014, now abandoned.
(60) Provisional application No. 61/861,958, filed on Aug. 2, 2013.
(60) Provisional application No. 61/862,933, filed on Aug. 6, 2013.

(51) Int. Cl. A61K 35/12; A61K 35/22; C12N 5/071; C12N 5/00

(52) U.S. Cl. CPC A61K 35/12; A61K 35/22; C12N 2533/54; C12N 2533/52; C12N 5/0687; C12N 5/0686; C12N 2533/90; C12N 5/0068

(57) ABSTRACT

Region-specific extracellular matrix (ECM) biomaterials are provided. Such materials include acellular scaffolds, sponges, solutions, and hydrogels suitable for stem cell culture.

### CROSS-REFERENCE TO RELATED APPLICATIONS

This application claims the benefit of U.S. Provisional Application No. 61/861,958, filed Aug. 2, 2013 and claims the benefit of U.S. Provisional Application No. 61/862,933, filed Aug. 6, 2013.

### STATEMENT REGARDING FEDERALLY SPONSORED RESEARCH

This invention was made with government support under grant number EB002520 awarded by the National Institutes of Health. The government has certain rights in the invention.

### BACKGROUND OF THE DISCLOSED SUBJECT MATTER

### Field of the Disclosed Subject Matter

The disclosed subject matter relates to biomaterials derived from healthy, diseased, or transgenic region-specific tissue extracellular matrix. Particularly, the presently disclosed subject matter relates to methods to isolate, decellularize, and process regions or anatomical features of various organs from various sources (including human and animal; fetal, juvenile, and adult; healthy, diseased, and transgenic) into various formats including: acellular scaffolds, sponges, hydrogels, and solutions. The presently disclosed subject matter further relates to such scaffolds, sponges, hydrogels, and solutions suitable for stem cell culture.

### Background

Extracellular matrix (ECM) provides cells with a scaffold with tissue-specific cues (molecular, structural, biomechanical) that mediate cell function. Stem cells reside on specialized ECM niches where they remain quiescent until needed, such as stem cells in the papilla region of the kidney. Currently it is not possible to re-create the complex environment of tissues such as the kidney using synthetic materials.

Accordingly, there remains a need for a medium that provides an environment suitable for the growth of stem cells for various tissues, such as the kidney.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

Native extracellular matrix (ECM) that is secreted and maintained by resident cells is of great interest for cell culture and cell delivery. As set forth below, specialized bioengineered niches for stem cells can be established using ECM-derived scaffolding materials. Although various embodiments refer to the kidney as an example, the methods and products set forth herein are applicable to various tissues. As an exemplary system, kidney is selected because of the high regional diversification of its tissue matrix. By preparing the ECM from three specialized regions of the kidney (cortex, medulla and papilla; the whole kidney, heart and bladder as controls) in three forms: (i) intact sheets of decellularized ECM, (ii) ECM hydrogels, and (iii) soluble ECM, it is shown how the structure and composition of ECM affect the function of kidney stem cells (with mesenchymal stem cells, or MSCs, serving as control). All three forms of the ECM regulate kidney stem cell (KSC) function, with differential structural and compositional effects. KSCs cultured on papilla ECM consistently display higher metabolic activity and differences in cell morphology, alignment, proliferation and structure formation as compared to cortex and medulla ECM, the effects not observed in corresponding MSC cultures. Thus, tissue- and region-specific ECM can provide an effective substrate for in vitro studies of therapeutic stem cells.

Similarly, tissues from other organs of various sources are processed into various formats. Specific regions or anatomical features of exemplary organs including the adrenal gland, bladder, blood vessel, brain, breast, bone, esophagus, heart, kidney, larynx, liver, lung, lymph node, muscle, parathyroid, pancreas, placenta, skin, small intestine, spleen, stomach, testes, thymus, thyroid, umbilical cord, and uterus are processed into various formats. These tissues are drawn from various sources including human and animal; fetal, juvenile, and adult; and healthy, diseased, and transgenic tissues. These materials are processed into formats including acellular scaffolds, sponges, hydrogels, and solutions.

To achieve these and other advantages and in accordance with the purpose of the disclosed subject matter, as embodied and broadly described, the disclosed subject matter includes a culture medium. In some embodiments, the culture medium includes a scaffold and the scaffold comprises a decellularized extracellular matrix. In some embodiments, the scaffold comprises a substantially planar sheet. In some embodiments, the decellularized extracellular matrix is selected from the group consisting of: adrenal gland extracellular matrix, bladder extracellular matrix, blood vessel extracellular matrix, brain extracellular matrix, breast extracellular matrix, bone extracellular matrix, esophagus extracellular matrix, heart extracellular matrix, kidney extracellular matrix, larynx extracellular matrix, liver extracellular matrix, lung extracellular matrix, lymph node extracellular matrix, muscle extracellular matrix, parathyroid extracellular matrix, pancreas extracellular matrix, placenta extracellular matrix, skin extracellular matrix, small intestine extracellular matrix, spleen extracellular matrix, stomach extracellular matrix, testes extracellular matrix, thymus extracellular matrix, thyroid extracellular matrix, umbilical cord extracellular matrix, and uterus extracellular matrix. In some embodiments, the decellularized extracellular matrix is a region-specific extracellular matrix. In some embodiments, the decellularized extracellular matrix is an organ-specific extracellular matrix. In some embodiments, the decellularized extracellular matrix is extracellular matrix of a region of an organ. In some embodiments, the organ is the kidney and the region is selected from the group consisting of: cortex, medulla, and papilla.

In another aspect of the present subject matter, a kit for making a culture medium is provided. The kit includes a solution and at least one reagent. The solution comprises decellularized extracellular matrix. At least one reagent is adapted to reconstitute the solution into a hydrogel. In some embodiments, the reagent comprises phosphate buffered saline or sodium hydroxide. In some embodiments, the decellularized extracellular matrix is selected from the group consisting of: adrenal gland extracellular matrix, bladder extracellular matrix, blood vessel extracellular matrix, brain extracellular matrix, breast extracellular matrix, bone extracellular matrix, esophagus extracellular matrix, heart extracellular matrix, kidney extracellular matrix, larynx extracellular matrix, liver extracellular matrix, lung extracellular matrix, lymph node extracellular matrix, muscle extracellular matrix, parathyroid extracellular matrix, pancreas extracellular matrix, placenta extracellular matrix, skin extracellular matrix, small intestine extracellular matrix, spleen extracellular matrix, stomach extracellular matrix, testes extracellular matrix, thymus extracellular matrix, thyroid extracellular matrix, umbilical cord extracellular matrix, and uterus extracellular matrix. In some embodiments, the decellularized extracellular matrix is a region-specific extracellular matrix. In some embodiments, the decellularized extracellular matrix is an organ-specific extracellular matrix. In some embodiments, the decellularized extracellular matrix is extracellular matrix of a region of an organ. In some embodiments, the organ is the kidney and the region is selected from the group consisting of: cortex, medulla, and papilla.

In other aspects of the present subject matter, culture media are provided. In some embodiments, the culture media include a hydrogel, the hydrogel comprising decellularized extracellular matrix. In some embodiments, the culture media include solubilized decellularized extracellular matrix. In some embodiments, the culture media include a sponge, the sponge comprising decellularized extracellular matrix.

In another aspect, the disclosed subject matter includes a method of creating a hydrogel. A portion of an organ is extracted. The organ portion is decellularized to yield extracellular matrix. The extracellular matrix is powdered to yield a powder. The powder is digested to yield a digest. The digest is reconstituted into a hydrogel.

It is to be understood that both the foregoing general description and the following detailed description are exemplary and are intended to provide further explanation of the disclosed subject matter claimed.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIGS. 1A-C illustrate the stem cell niche of the kidney.

FIGS. 2A-F illustrate removal of cellular material and preservation of ECM in decellularized kidney regions.

FIGS. 3A-B illustrate ultrastructure of native and decellularized kidney regions.

FIGS. 4A-B illustrate collagen IV and fibronectin native and decellularized kidney regions.

FIGS. 5A-F illustrate DNA and metabolic activity of KSCs and MSCs in the presence of solubilized regionally specific kidney ECM.

FIGS. 6A-H illustrate metabolic activity, DNA content, and rhodamine-phalloidin/DAPI staining of KSCs and MSCs on regional kidney ECM hydrogels.

FIGS. 7A-H illustrates metabolic activity, DNA content, and rhodamine-phalloidin/DAPI staining of KSCs and MSCs on region-specific decellularized kidney ECM sheets.

FIG. 8 illustrates live/dead and rhodamine/phalloidin staining of KSCs and MSCs on regionally specific decellularized kidney region ECM sheets.

FIGS. 9A-D illustrate organ-specific effects of ECM on metabolism of kidney stem cells.

FIGS. 10A-C illustrate characterization of solubilized kidney region ECM and ECM hydrogels.

FIGS. 11A-B illustrate chemotaxis of KSCs in the presence of solubilized kidney region ECM.

### DETAILED DESCRIPTION OF EXEMPLARY EMBODIMENTS

Reference will now be made in detail to exemplary embodiments of the disclosed subject matter. Methods and corresponding steps of the disclosed subject matter will be described in conjunction with the detailed description of the system.

The extracellular matrix (ECM), the native scaffolding material secreted and maintained by residents cells, provides an ideal microenvironment for the cells with tissue-specific physical and molecular cues mediating cell proliferation, differentiation, gene expression, migration, orientation, and assembly. Functional and structural components within the ECM contribute to the extracellular environment specific to each tissue and organ. The complexity of the ECM has proven difficult to recapitulate in its entirety. Mimicking just the ECM structure using synthetic biomaterials or mimicking composition by adding purified ECM components is possible. While offering structural mimics, synthetic biomaterials can potentially generate cytotoxic by-products at the site of implantation, leading to poor wound healing or an inflammatory environment.

An alternative to synthetic biomaterials is to directly isolate the native ECM from the tissue of interest via the removal of cells and cellular remnants. ECM scaffolds may be derived from a variety of tissues such as adrenal gland, bladder, blood vessel, brain, breast, bone, esophagus, heart, kidney, larynx, liver, lung, lymph node, muscle, parathyroid, pancreas, placenta, skin, small intestine, spleen, stomach, testes, thymus, thyroid, umbilical cord, and uterus. ECM-derived biomaterials can be processed into scaffolds (such as acellular scaffolds or sponges) with appropriate compositions and structures for cell cultivation and tissue engineering. Furthermore, ECM scaffolds gradually degrade while promoting tissue remodeling at the site of implantation. Due to their biocompatibility and their ability to modulate the host tissue response, ECM scaffolds are suitable for tissue engineering and regenerative medicine applications. ECM scaffolds can be derived from various sources such as human and animal; fetal, juvenile, and adult; healthy, diseased, and transgenic tissues.

ECM-based scaffolds can also be used to regulate the differentiation and maintenance of stem cells and their differentiated progeny. Stem cells normally reside within unique and highly regulated ECM serving as a niche. Complex tissues such as heart and lung may be subjected to decellularization to obtain native-ECM scaffolds without particular regard for any specific region of the organ or preservation of potential stem cell niches. However, cells native to a particular region of the organ (e.g., vascular endothelium, liver sinusoidal cells) display ECM recognition and specificity. Extending this site-specific recognition to stem cells renders the choice of matrix an important consideration.

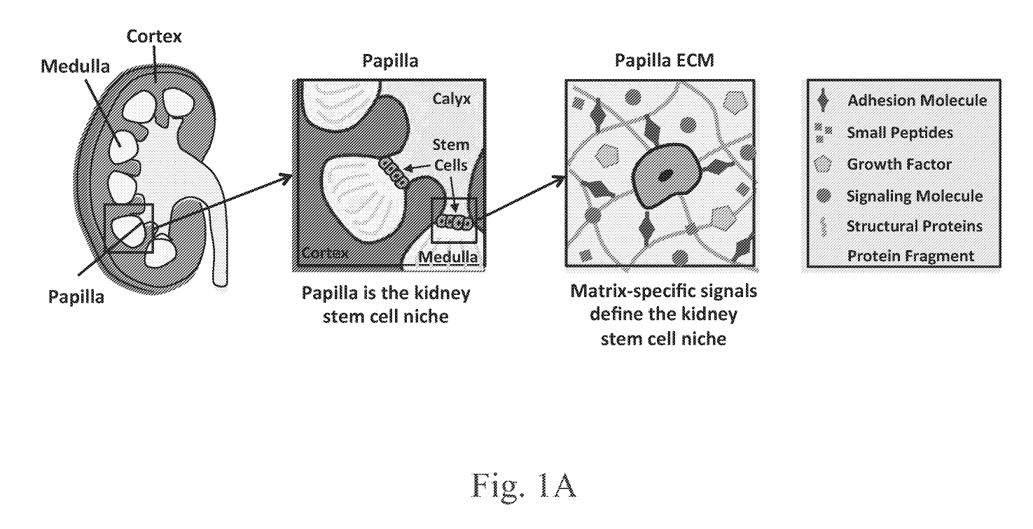

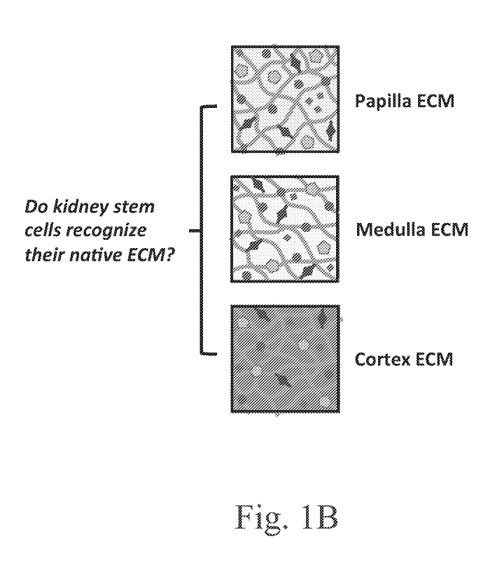

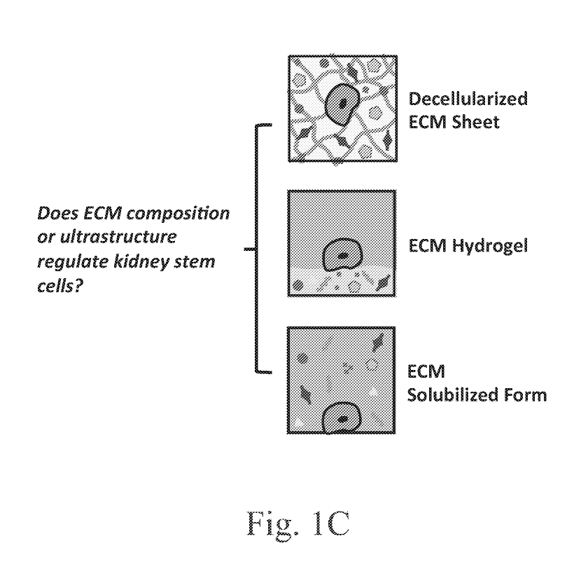

Referring to FIG. 1, the stem cell niche of the kidney is illustrated. In FIG. 1A: The renal papilla is the stem cell niche in the kidney, defined by region-specific cues in the extracellular matrix of the papilla. In FIG. 1B: The renal cortex, medulla, and papilla were dissected and decellularized separately to obtain region-specific kidney ECM. In FIG. 1C: ECM was prepared in sheets, hydrogels, and solubilized form for cultivation of the two types of stem cells (kidney stem cells and mesenchymal stem cells). To obtain regionally specific kidney ECM sheets, kidney regions can be dissected before decellularization or the regional matrix is punched from whole decellularized kidney sections. Alternatively, pre-sectioned regions were decellularized, snap-frozen in liquid nitrogen, ground into a fine powder, lyophilized, and pepsin-digested to yield regionally specific kidney ECM digests that were neutralized to obtain solubilized ECM forms. By treating these digests with salt, base, and heat, we obtained regionally specific kidney ECM hydrogels. Kidney stem cells (KSCs) that are native to the papilla were cultured on ECM sheets, hydrogels, or solubilized forms derived from the three kidney regions (cortex, medulla, papilla) and compared to mesenchymal cells (MSCs) cultured under the same conditions.

The kidney is a suitable organ for studying effects of regional ECM on the resident stem cell population. A cross-sectional view of the kidney reveals three distinct regions: cortex, medulla, and papilla (FIG. 1A), with each region displaying its unique structure, function, and composition, and residing in environments with very different osmolalities and oxygen tensions.

The cortex contains renal corpuscles, and the associated convoluted and straight tubules, collecting tubules and ducts, and contains an extensive vascular network. The medulla is arranged into pyramids, and characterized by straight tubules, collecting ducts, and the vasa recta, a specialized capillary system involved in the concentration of urine. At the apex of each medullary pyramid, where the collecting ducts converge and empty into the renal calyx, is the papilla. The renal papillae contain a putative population of adult stem cells that remains quiescent after the development is complete and is mobilized again during injury. This stem cell may be isolated and expanded in culture, making the kidney an excellent model to study interactions between the native stem cell population and the matrix derived from distinct regions within the organ.

ECM materials according to various embodiments of the present disclosure are useful to grow, maintain, or differentiate organ- or region-specific cells in culture. Various embodiments of the present disclosure are useful for: in vitro three-dimensional culture and testing of cells on acellular scaffolds, in sponges, or in hydrogels; in vitro culture and testing of cells with culture medium supplemented with matrix solution; in vitro coating (adsorption) of matrix solution to cell culture flask/dish to increase attachment, growth; in vitro guided differentiation of embryonic stem cells or induced pluripotent stem cells into organ-specific cells; in vivo injection/delivery of therapeutic cells, drugs, or other soluble factors via hydrogel; in vivo implantation of acellular scaffolds with or without cells for tissue/organ regeneration studies.

The present disclosure describes a method to derive regionalized ECM biomaterials, for example, for stem cell culture. Such materials include acellular scaffolds, sponges, hydrogels, and solutions. According to various embodiments of the present disclosure, materials are provided in various physical forms including various sized sheets and solubilized forms. According to various embodiments, ECM biomaterials are derived from various tissues including adrenal gland, bladder, blood vessel, brain, breast, bone, esophagus, heart, kidney, larynx, liver, lung, lymph node, muscle, parathyroid, pancreas, placenta, skin, small intestine, spleen, stomach, testes, thymus, thyroid, umbilical cord, and uterus. In some embodiments, region-specific ECM biomaterials are derived from a corresponding region in a source organ, for example, from the cortex, medulla or papilla of a kidney. Tissues sources include human and animal; fetal, juvenile, and adult; healthy, diseased, and transgenic. Described below are the regionally specific effects of kidney ECM on the growth and metabolism of kidney stem cells, how these effects depend on the preservation of ECM structure vs only composition, and extension of these effects to exogenous (non-kidney) stem cells, such as mesenchymal stem cells (MSCs).

Overview

The methods and systems presented herein may be used for creating ECM biomaterials for stem cell culture from various tissues, including region-specific kidney extracellular matrix hydrogels. The disclosed subject matter is particularly suited for creating region-specific hydrogels for the growth of stem cells, such as kidney stem cells (KSCs), and mesenchymal stem cells (MSCs).

According to embodiments of the present disclosure, native tissue matrix is used to cause region-specific effects on the growth of KSCs and mesenchymal stem cells (MSCs). To this end, hydrogels are derived from kidney regions including the cortex, medulla and papilla.

According to an exemplary method, kidneys are procured and immediately frozen and prepared for sectioning. Frozen blocks are then sectioned longitudinally into thin (200 μm-1 mm) slices showing the entire cross-section of the kidney. The cortex, medulla, and papillae of the kidney are then dissected and separated from the thin slices prior to decellularization.

The tissues are decellularized using a 4-step method consisting of 0.02% trypsin (2 hr), 3% Tween-20 (2 hr), 4% sodium deoxycholate (2 hr), and 0.1% peracetic acid (1 hr). Each step is followed by deionized water and 2×PBS washes. In some embodiments, each region is decellularized by serial washes in 0.02% trypsin, 3% Tween, 4% deoxycholic acid, and 0.1% peracetic acid solutions followed by enzymatic digestions.

Following decellularization, the ECMs are snap frozen in liquid nitrogen, pulverized using a mortar and pestle, and then lyophilized to obtain a fine powder. Lyophilized ECM powder is digested using pepsin and hydrochloric acid for 48 hours at room temperature. The resulting digest is re-constituted into a hydrogel by increasing the ionic strength and the pH of the solution using PBS and NaOH.

The results are highly specific hydrogels composed of the native extracellular matrix surrounding native cells. They may be used to grow and maintain tissue-specific cells in culture. In some embodiments, cells are cultured on the hydrogels. In other embodiments, cells are cultured in media supplemented with digested ECM. Metabolic activity, image analysis and DNA quantification may be performed.

In one embodiment, kidney stem cells isolated from the papilla are maintained by culturing the cells in papilla derived ECM hydrogels in vitro. Hydrogels may also be used as an injectable therapeutic platform for the delivery of drugs and/or cell therapy to an injured kidney or to guide the differentiation of embryonic stem cells or induced pluripotent stem cells into kidney specific cells for renal tissue engineering applications.

KSCs cultured in the presence of papilla ECM show higher metabolic activity and lower DNA content when compared to whole kidney, cortex and medulla ECM, an effect not observed using MSCs. Thus, the hydrogels derived from the native kidney ECM stimulate the parent KSCs but not the MSCs. Region specific kidney ECM affects the growth and metabolism of KSCs. Region-specific ECM thus provides a suitable substrate for cultivation and delivery of stem cells and their derivatives.

According to various embodiments of the present disclosure, ECM is extracted from organs and tissues including the adrenal gland, bladder, blood vessel, brain, breast, bone, esophagus, heart, kidney, larynx, liver, lung, lymph node, muscle, parathyroid, pancreas, placenta, skin, small intestine, spleen, stomach, testes, thymus, thyroid, umbilical cord, and uterus. Organs/tissues are procured, prepared for sectioning, frozen, then sectioned into thin slices. In some embodiments, the slices are about 200 μm to about 1 mm thick. Organ regions, sub-sections, or anatomical features of interest are further dissected and separated prior to decellularization. In various exemplary embodiments, region-specific tissues are extracted from the kidney cortex, medulla, or papilla; the lung airways or parenchyma; the esophageal endomucosa or muscularis externa; or the heart ventricle or atrium.

Tissue sections are decellularized by the introduction of one or more of deionized water, hypertonic salines, enzymes, detergents, and acids. In an exemplary embodiment, heart ventricle sections are decellularized by 0.02% trypsin (2 hr), 3% Tween-20 (2 hr), 4% sodium deoxycholate (2 hr), and 0.1% peracetic acid (1 hr). Each step is followed by deionized water and hypertonic (2×) phosphate-buffered saline (PBS) washes. Exemplary embodiments for various organs and tissues of human and animal origin are provided below in Table 1.

TABLE 1

| Organ | Step 1 | Step 2 | Step 3 | Step 4 | Step 5 | Step 6 |
|---|---|---|---|---|---|---|
| Bladder | Trypsin, | Tween-20, | Sodium | Peracetic | | |
| | 0.02%, 60 min | 3%, 120 min | Deoxycholate, | Acid, 0.1%, | | |
| | | | 4%, 120 min | 30 min | | |
| Bone | Tween-20, | CHAPS, | Sodium | Peracetic | | |
| | 3%, 120 min | 8 mM, 120 min | Deoxycholate, | Acid, 0.1%, | | |
| | | | 4%, 120 min | 15 min | | |
| Brain | Tween-20, | Tween-20, | Sodium | | | |
| | 3%, 60 min | 3%, 60 min | Deoxycholate, | | | |
| | | | 4%, 30 min | | | |
| Esophagus | Trypsin, | Tween-20, | Tween-20, | Sodium | Sodium | Peracetic |
| | 0.02%, 60 min | 3%, 120 min | 3%, 120 min | Deoxycholate, | Deoxycholate, | Acid, 0.1%, |
| | | | | 4%, 120 min | 4%, 120 min | 60 min |
| Heart | Trypsin, | Tween-20, | Sodium | Peracetic | | |
| | 0.02%, 120 min | 3%, 120 min | Deoxycholate, | Acid, 0.1%, | | |
| | | | 4%, 120 min | 30 min | | |
| Kidney | Trypsin, | Tween-20, | Sodium | Peracetic | | |
| | 0.02%, 120 min | 3%, 120 min | Deoxycholate, | Acid, 0.1%, | | |
| | | | 4%, 120 min | 60 min | | |
| Liver | Deionized | Tween-20, | Deionized | Sodium | Deionized | |
| | water | 3%, 180 min | water | Deoxycholate, | water | |
| | | | | 4%, 180 min | | |
| Lung | Trypsin, | CHAPS, | CHAPS, | Peracetic | | |
| | 0.02%, 60 min | 8 mM, | 8 mM, | Acid, 0.1%, | | |
| | | 120 min | 120 min | 60 min | | |
| Muscle | Trypsin, | Tween-20, | Sodium | Peracetic | | |
| | 0.02%, 120 min | 3%, 120 min | Deoxycholate, | Acid, 0.1%, | | |
| | | | 4%, 120 min | 60 min | | |
| Pancreas | Deionized | Tween-20, | Deionized | Sodium | Deionized | |
| | water | 3%, 180 min | water | Deoxycholate, | water | |
| | | | | 4%, 180 min | | |
| Placenta | Deionized | Tween-20, | Deionized | Sodium | Deionized | Peracetic |
| | water | 3%, 180 min | water | Deoxycholate, | water | Acid, 0.1%, |
| | | | | 4%, 180 min | | 60 min |
| Skin | Trypsin, | Tween-20, | Sodium | Peracetic | | |
| | 0.02%, 60 min | 3%, 60 min | Deoxycholate, | Acid, 0.1%, | | |
| | | | 4%, 60 min | 60 min | | |
| Small | Trypsin, | CHAPS, | CHAPS, | Peracetic | | |
| Intestine | 0.02%, 60 min | 8 mM, | 8 mM, | Acid, 0.1%, | | |
| | | 120 min | 120 min | 60 min | | |
| Spleen | Trypsin, | Tween-20, | Sodium | Peracetic | | |
| | 0.02%, 120 min | 3%, 120 min | Deoxycholate, | Acid, 0.1%, | | |
| | | | 4%, 120 min | 60 min | | |
| Stomach | Trypsin, | Tween-20, | Tween-20, | Sodium | Sodium | Peracetic |
| | 0.02%, 60 min | 3%, 120 min | 3%, 120 min | Deoxycholate, | Deoxycholate, | Acid, 0.1%, |
| | | | | 4%, 120 min | 4%, 120 min | 60 min |
| Thymus | Tween-20, | Tween-20, | Sodium | | | |
| | 3%, 120 min | 3%, 120 min | Deoxycholate, | | | |
| | | | 4%, 60 min | | | |
| Umbilical | Trypsin, | Tween-20, | CHAPS, | Peracetic | | |
| Cord | 0.02%, 15 min | 3%, 120 min | 8 mM, 60 min | Acid, 0.1%, | | |
| | | | | 30 min | | |
| Vessel | Tween-20, | CHAPS, | | | | |
| (e.g., | 3%, 120 min | 8 mM, 60 min | | | | |
| Inferior | | | | | | |
| Vena Cava) | | | | | | |

Following decellularization, resulting materials are terminally sterilized and biopsied according to desired scaffold size. In some embodiments, the scaffold is sized to fit in the wells of a standard a microtiter plate, for example a 24- or 96-well plate.

In some embodiments, following decellularization, an ECM solution is produced. The decellularized material is snap frozen in liquid nitrogen, pulverized using a mortar and pestle, lyophilized, and then milled to obtain a fine ECM powder. In some embodiments, the ECM powder is digested using 1 mg/mL pepsin and 0.1M hydrochloric acid for more than 24 hrs at room temperature. The resulting digest is neutralized, frozen, and thawed to obtain ECM solution.

In some embodiments, ECM powder is further processed to form an ECM sponge. ECM powder is digested using 1 mg/mL pepsin and 0.1M hydrochloric acid for less than 24 hrs at room temperature. The resulting digest is subjected to repeated cycles of high-speed centrifugation (5,000 rpm) and vortexing. The resulting material is transferred to a mold of desired dimensions and lyophilized. The resulting sponge can be sectioned, re-sized, or re-hydrated. In some embodiments, the sponge is sized to fit in the wells of a standard a microtiter plate, for example a 24- or 96-well plate.

In some embodiments, ECM solution is ECM solution is re-constituted into a hydrogel by increasing the ionic strength and the pH of the solution using PBS and NaOH.

Composition and Gelation Properties of Decellularized Kidney ECM

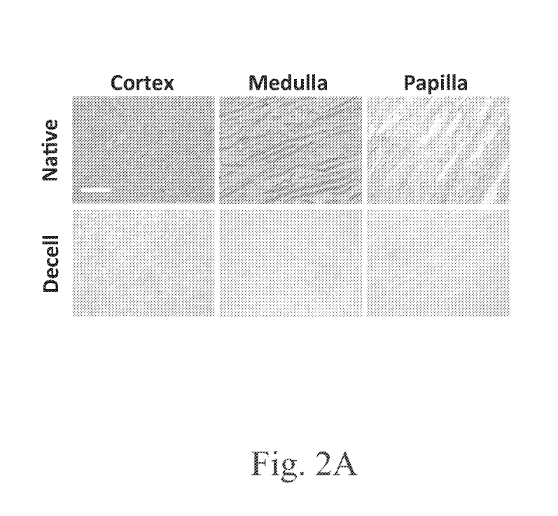

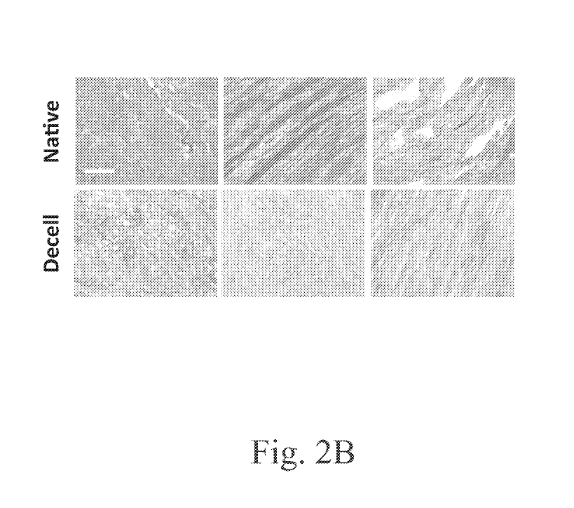

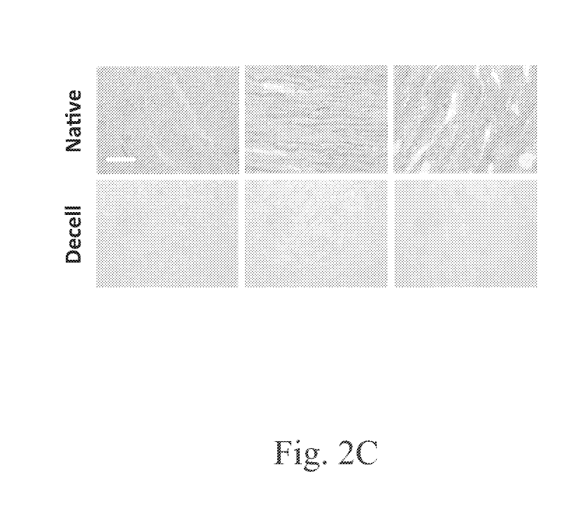

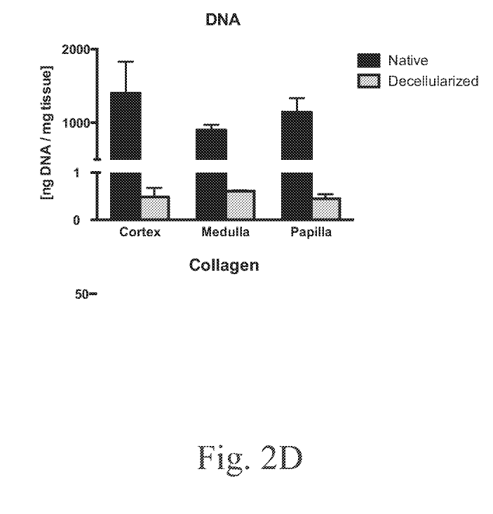

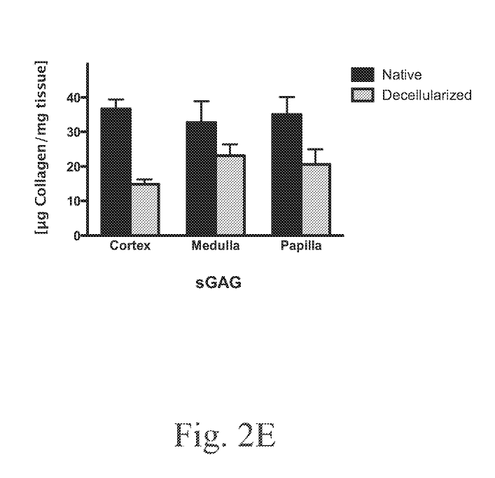

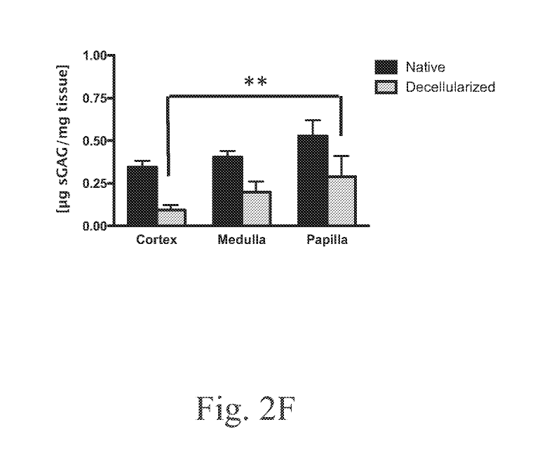

Referring to FIG. 2, removal of cellular material and preservation of ECM in decellularized kidney regions is illustrated. Histology confirms decellularization with preservation of matrix proteins in kidney regions. In FIG. 2A: H&E stain shows the absence of cell nuclei. In FIG. 2B: Trichrome stain shows the preservation of collagen (blue), and in FIG. 2C: Alcian Blue stain shows loss of proteoglycans (light blue). In FIG. 2D: DNA quantification indicates >99% removal of nuclear material after decellularization. In FIG. 2E: Collagen quantification shows comparable retention of collagen among kidney regions. In FIG. 2F: Sulfated glycosaminoglycan (sGAG) quantification indicates papilla retains significantly more sGAG than cortex.

Decellularization of kidney regions (cortex, medulla, papilla) by a four step method (trypsin, Tween 20, sodium deoxycholate, peracetic acid) resulted in the removal of >99% nuclear material as shown by H&E staining and DNA quantification (FIGS. 2A, 2D). Collagen content of decellularized kidney regions was reduced in all three regions, and most significantly in the cortex (FIGS. 2B, 2E). A similar trend was observed with sulfated glycosaminoglycans (sGAG) content (FIGS. 2C, 2F). Histological sections of kidney regions stained with H&E (FIG. 2A), Trichrome (FIG. 2B), and Alcian Blue (FIG. 2C) show complete removal of cellular nuclei with some preservation of ECM structure and distribution of remaining collagen (blue) (FIG. 2B) and glycosaminoglycans (blue) (FIG. 2C).

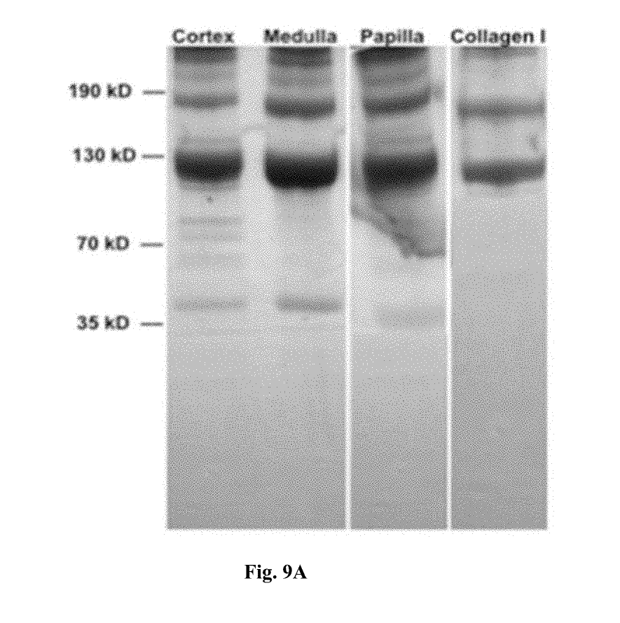

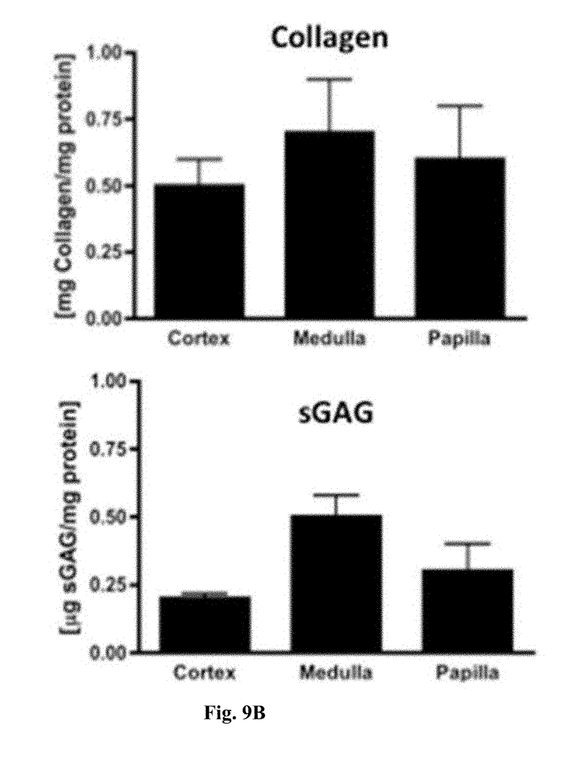

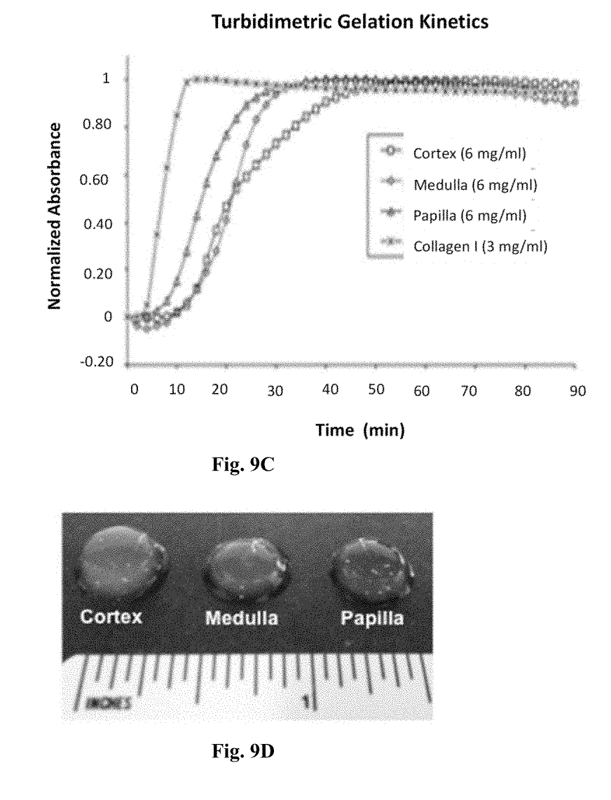

Electrophoresed kidney region ECM digests and purified collagen I showed major bands at similar locations, indicating that collagen I is a large component of the kidney region ECM digests, with other bands distinct from pure collagen I (FIG. 9A). In the digested (solubilized) ECM from kidney regions, the amounts of collagen per unit ECM protein were comparable among the three regions, while the amount if sGAG per unit ECM protein was lower for the cortex region (FIG. 9B). The measurements of gelation kinetics showed sigmoidal curves for ECM derived from all kidney regions and the collagen I hydrogel, with kidney hydrogels having delayed kinetics relatively to collagen I (FIG. 9C). Among the kidney hydrogels, the time for gelation increased from papilla to medulla and cortex. Polymerized kidney region hydrogels had similar macroscopic appearance (FIG. 9D).

Ultrastructure of Native and Decellularized Kidney ECM

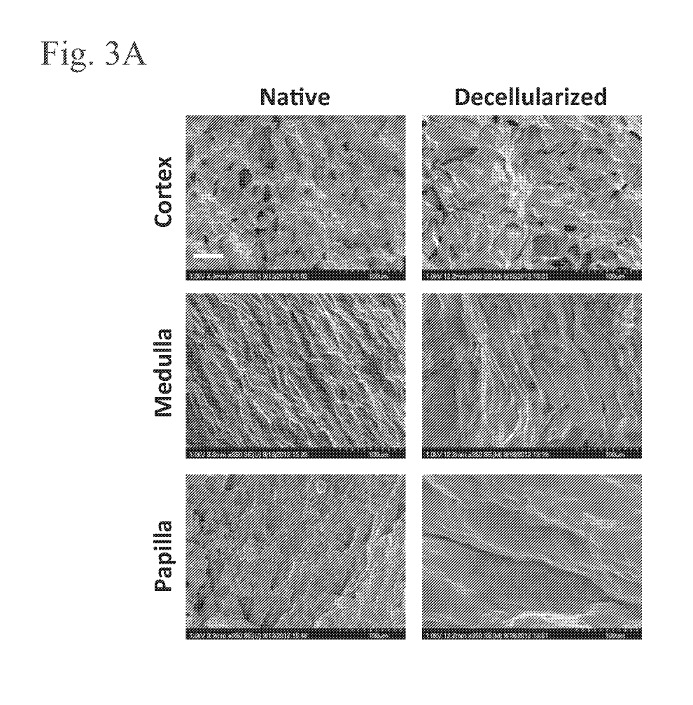

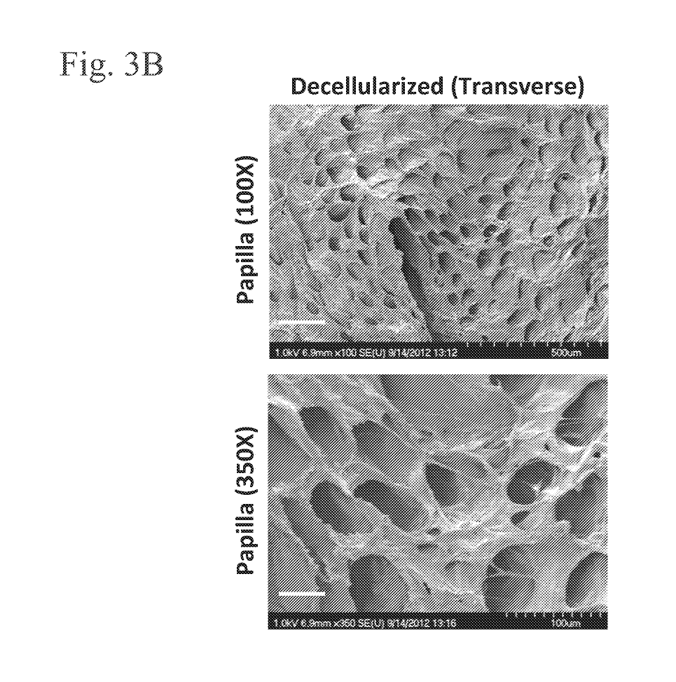

Referring to FIG. 3, ultrastructure of native and decellularized kidney regions is illustrated. In FIG. 3A, Scanning electron microscopy at 350X reveals differences in ECM topography between kidney regions before and after decellularization. In FIG. 3B, Transverse sections of decellularized papilla at 100× and 350× indicate that tubular ultrastructure is preserved in the KSC niche after decellularization.

Native and decellularized kidney regions were imaged via SEM to investigate preservation of the ultrastructure after decellularization (FIG. 3). Increased magnification at 350X reveals large differences in the native structures of the ECM in various regions of the kidney as well as distinct topographical differences retained in decellularized kidney regions (FIG. 3A). A transverse section of decellularized papilla reveals preservation of tubular ultrastructure in the KSC niche (FIG. 3B).

Collagen IV and Fibronectin in Native and Decellularized Kidney ECM

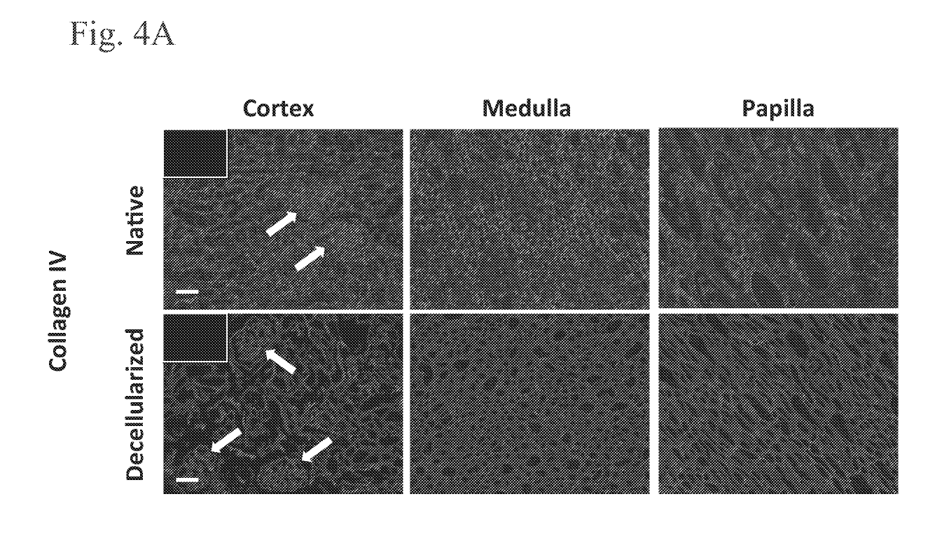

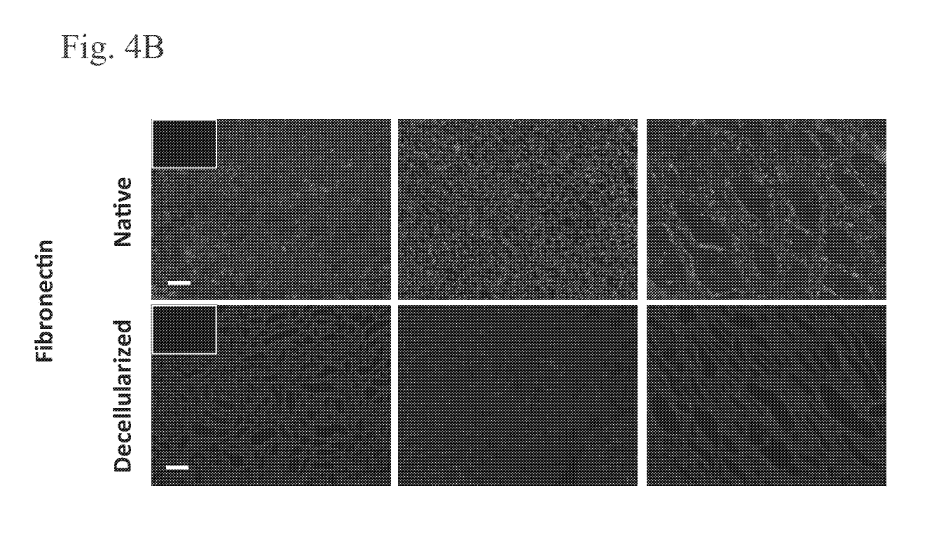

Referring to FIG. 4, Collagen IV and fibronectin native and decellularized kidney regions are illustrated. Immunostaining reveals in FIG. 4A significant retention of Collagen IV in the basement membrane of decellularized kidney regions, including preservation of glomerular structures in decellularized cortex (arrows), and in FIG. 4B depletion of fibronectin in kidney regions after decellularization.

Native and decellularized kidney regions were immunostained to reveal the amounts and distributions of collagen IV and fibronectin in kidney regions before and after decellularization (FIG. 4). Immunostaining for collagen IV indicates a significant amount of Collagen IV is retained after decellularization as well as the retention of renal corpuscular structures in the cortex (FIG. 4A). Immunostaining for fibronectin indicates a significant loss of fibronectin after decellularization (FIG. 4B).

DNA and Metabolic Activity of KSCs in Solubilized Kidney ECM

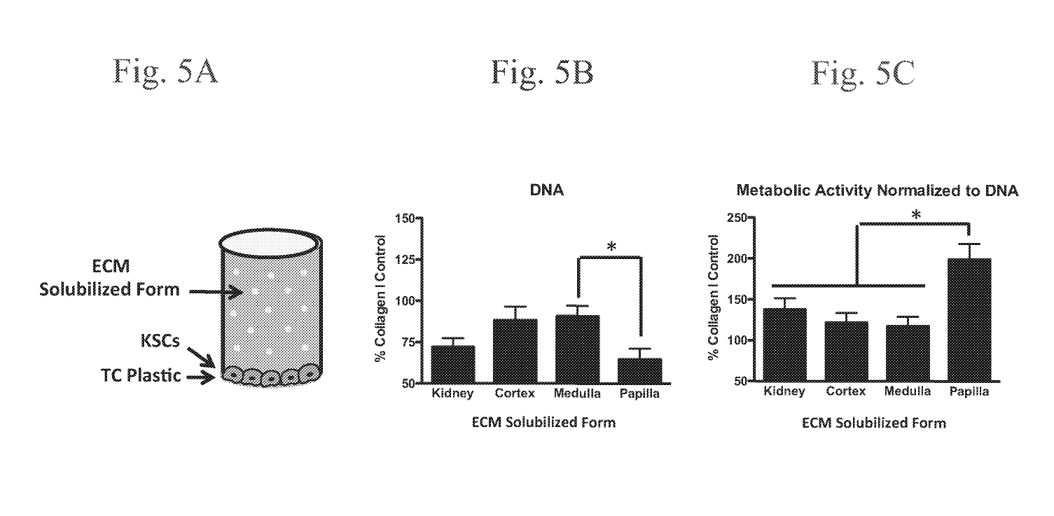

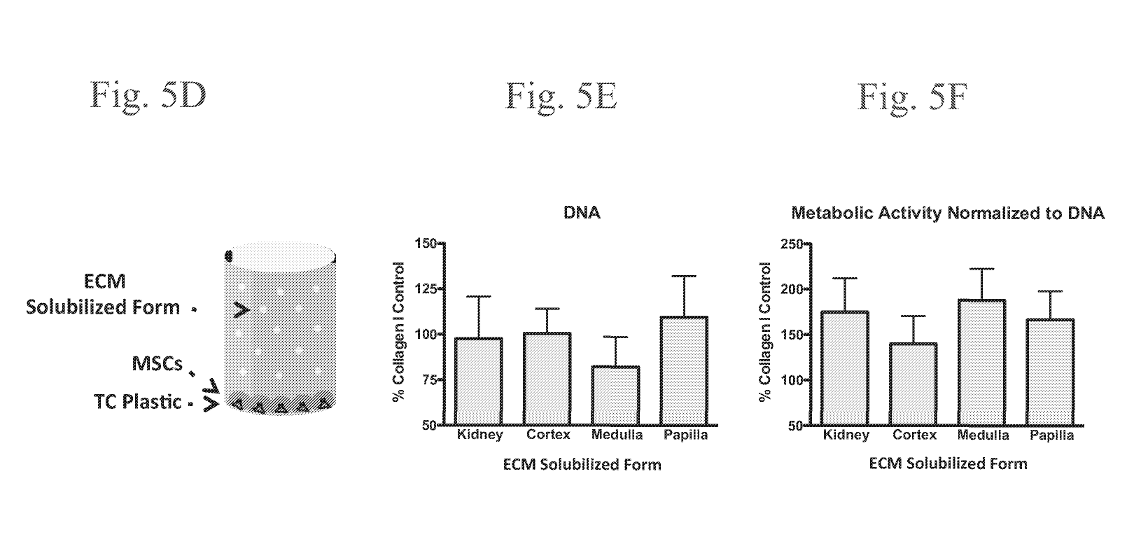

Referring to FIG. 5, DNA and metabolic activity of KSCs and MSCs in the presence of solubilized regionally specific kidney ECM is illustrated. In FIG. 5A, KSCs were cultured on tissue culture plastic in the presence of solubilized whole organ or regional kidney ECM. In FIG. 5B, DNA quantification of KSCs revealed less DNA in the presence of solubilized papilla ECM than in the presence of solubilized ECM from cortex, medulla, or whole kidney. In FIG. 5C, KSCs cultured in the presence of solubilized papilla ECM were significantly more metabolically active than KSCs cultured in the presence of solubilized ECM from cortex, medulla, or whole kidney. In FIG. 5D, MSCs cultured on tissue culture plastic in the presence of solubilized whole kidney and kidney region ECM. In FIG. 5E, DNA quantification revealed no significant differences between MSCs cultured in the presence of whole organ or regional solubilized kidney ECM. In FIG. 5F, metabolic activity of MSCs showed no differences between MSCs cultured in the presence of whole organ or regional solubilized kidney ECM.

KSCs and MSCs were cultured on tissue culture plastic in media supplemented with solubilized ECM derived from the three kidney regions or the whole kidney. DNA and metabolic activity were measured and expressed relatively to the corresponding values measured for cells grown in media supplemented with purified solubilized collagen I (FIGS. 5A, 5D). The number of KSCs in cultures with solubilized papilla ECM was significantly lower than in the solubilized ECM from any other region or the whole kidney (FIG. 5B), indicating that papillary ECM suppresses cell cycling. The whole kidney ECM showed an intermediate value between the ECMs of the three regions. Metabolic activity per unit DNA indicates that KSCs grown in solubilized papilla, although fewer in number, were significantly more metabolically active than KSCs grown in solubilized whole kidney, cortex, or medulla ECM.

No significant differences in metabolism were observed between KSCs in solubilized cortex and medulla ECM (FIG. 5C). MSCs in solubilized kidney region ECM showed no significant differences in DNA or metabolic activity (FIGS. 5E, 5F).

DNA, Metabolic Activity, and Phenotype of KSCs on Regional Kidney ECM Hydrogels

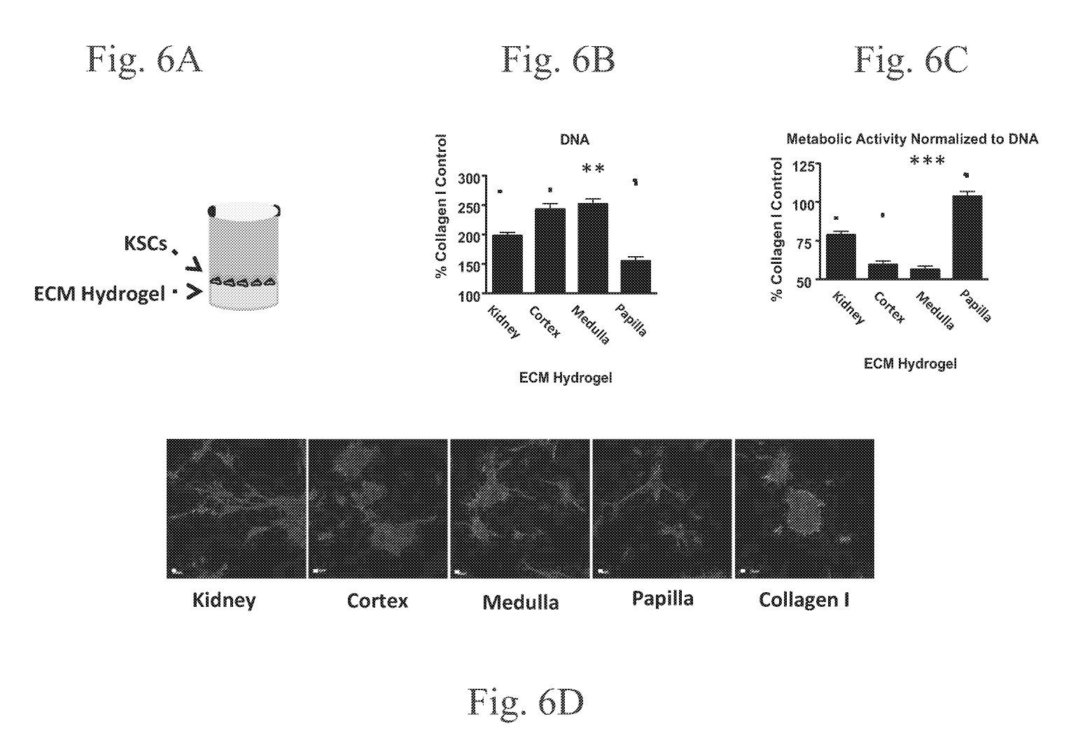

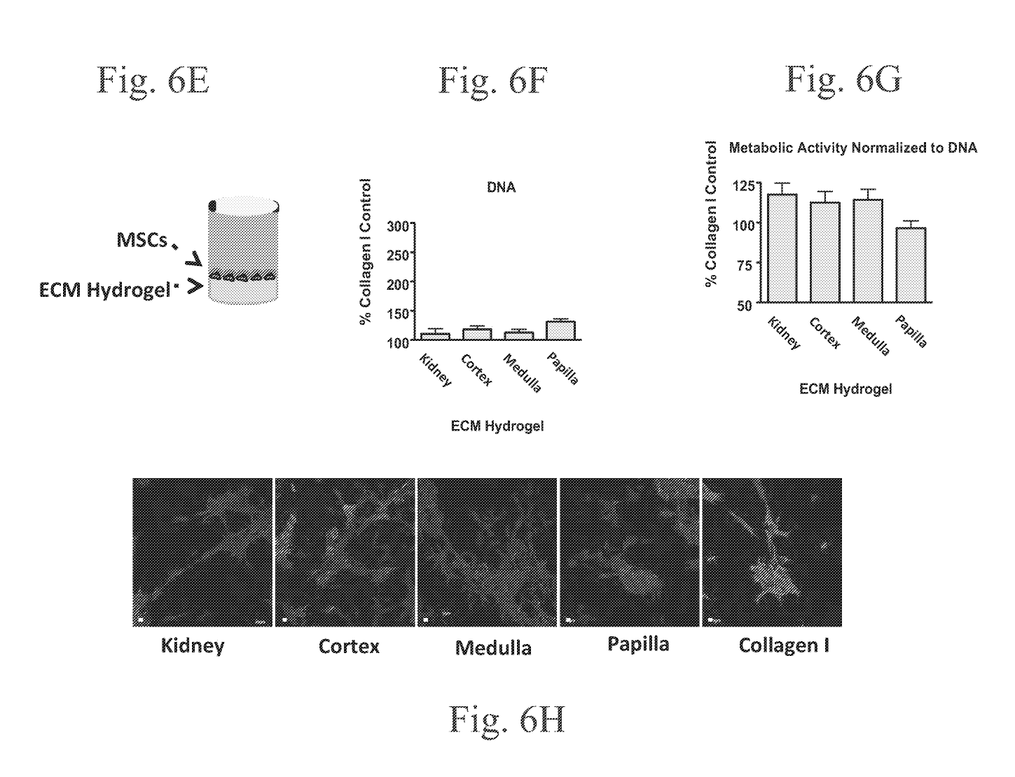

Referring to FIG. 6, metabolic activity, DNA content, and rhodamine-phalloidin/DAPI staining of KSCs and MSCs on regional kidney ECM hydrogels are illustrated. In FIG. 6A, KSCs seeded onto ECM hydrogels and cultured for 48 hrs. In FIG. 6B, DNA quantification of KSCs shows that papilla hydrogel contained significantly fewer cells than EXM derived from other kidney regions or the whole kidney. In FIG. 6C, metabolic activity normalized to DNA reveals that KSCs are significantly more metabolically active on papilla ECM hydrogel than on ECM hydrogels from other kidney regions or the whole kidney. In FIG. 6D, confocal imaging of KSCs on regional kidney ECM hydrogels and Collagen I hydrogel shows longitudinal cell alignment in kidney ECM hydrogels but not in Collagen I. In FIG. 6E, MSCs seeded onto ECM hydrogels and cultured for 48 hrs. In FIG. 6F, DNA quantification of MSCs also shows no significant differences between regional kidney hydrogels. In FIG. 6G, Metabolic activity per unit DNA for MSCs was comparable for all ECM hydrogels, derived regionally or from the whole kidney. In FIG. 6H, Confocal imaging of MSCs on kidney region hydrogels and Collagen I shows similar cell morphology in all kidney ECM hydrogels and Collagen I hydrogel. Scale bars: 50 μm.

KSCs and MSCs were seeded at equal densities on decellularized kidney ECM hydrogels (FIGS. 6A, 6E), cultured for 48 hrs, assayed for DNA and metabolic activity, and data were normalized to those measured for collagen I hydrogel. DNA quantification of KSCs revealed significant differences between whole kidney, cortex, medulla, and papilla regions, with papilla hydrogel again yielding significantly fewer KSCs (FIG. 6B). The whole kidney hydrogel showed an intermediate value approximating an average for the three regions. Equal initial seeding densities resulted in significantly more KSCs on kidney region ECM hydrogels than MSCs after 48 hrs (FIGS. 6B, 6F).

Metabolic activity per unit DNA again indicated that KSCs on papilla ECM hydrogel were significantly more metabolically active than were KSCs on whole kidney, cortex or medulla ECM hydrogel. No significant differences were observed in metabolism between KSCs on cortex and medulla hydrogels (FIG. 6C). Morphology of KSCs on whole kidney and regional kidney hydrogels appeared consistent (FIG. 6D). No significant differences in DNA, metabolic activity, or morphology were observed for MSCs on kidney region ECM hydrogels (FIGS. 6F, 6G, 6H).

DNA, Metabolic Activity, and Phenotype of KSCs on Regional Kidney ECM Sheets

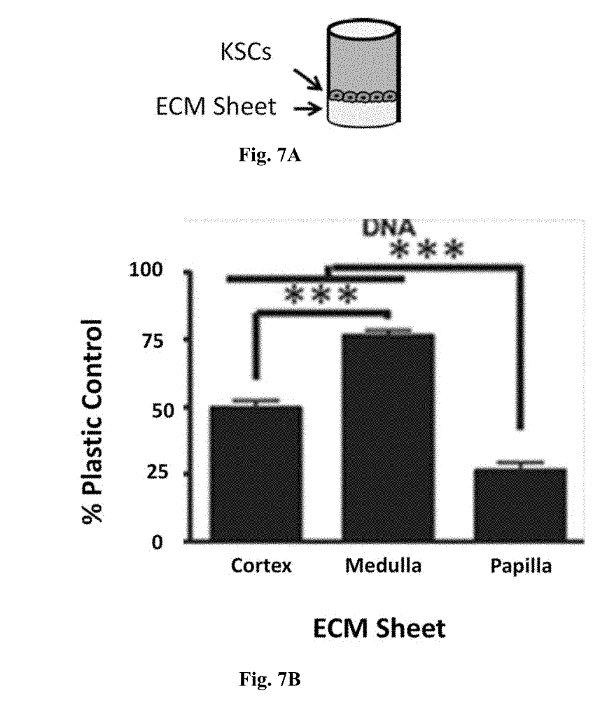

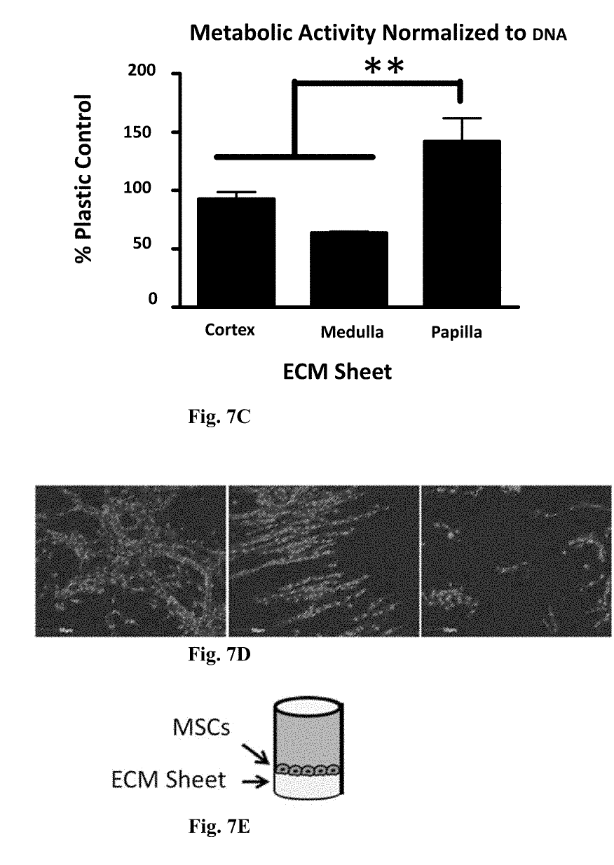

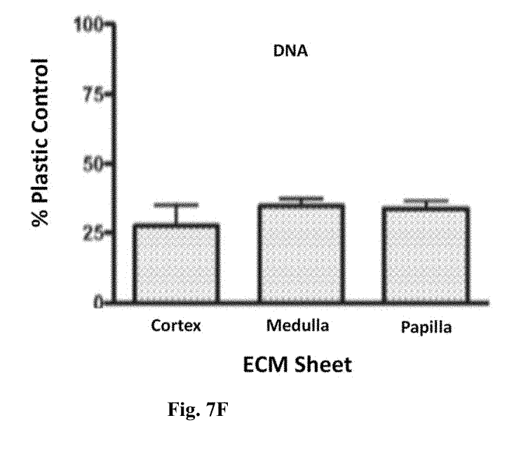

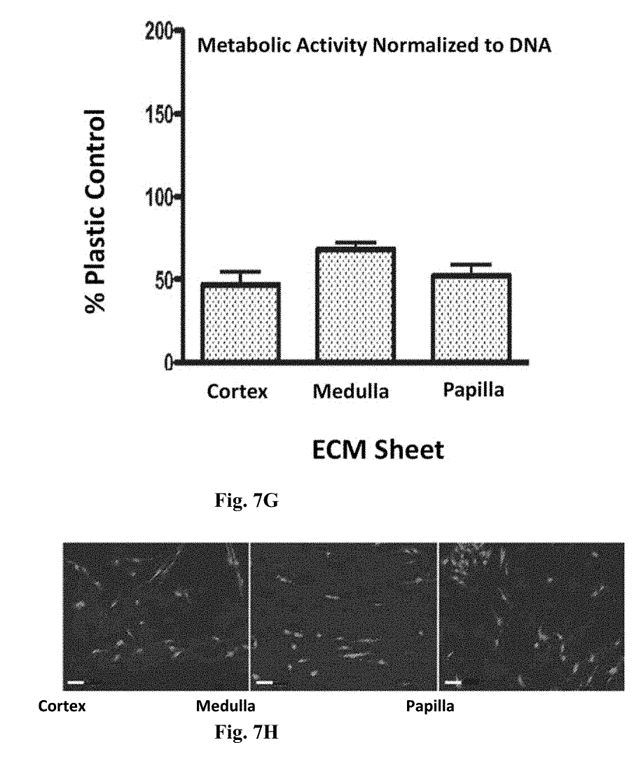

Referring to FIG. 7, Metabolic activity, DNA content, and rhodamine-phalloidin/DAPI staining of KSCs and MSCs on region-specific decellularized kidney ECM sheets are illustrated. In FIG. 7A, KSCs seeded onto ECM sheets and cultured for 48 hrs. In FIG. 7B, DNA quantification reveals that papilla ECM contained significantly fewer cells, and that medulla contained significantly more cells than either cortex or papilla. In FIG. 7C, Metabolic activity per unit DNA indicates that the fewer KSCs on papilla are significantly more metabolically active than KSCs on cortex or medulla ECM. In FIG. 7D, Rhodamine-Phalloidin/DAPI staining shows clear differences in morphology, orientation, and structure formation between KSCs on cortex, medulla, and papilla ECM sheets. KSCs on cortex show star-like morphology with random orientation, whereas KSCs on medulla exhibit elongated morphology with significant aligning and the formation of tubular structures. KSCs on papilla show clusters with periodic rounded morphology. In FIG. 7E, MSCs are seeded onto ECM sheets and cultured for 48 hrs. F DNA quantification shows no significant differences in MSC cultures on ECM from different kidney regions. In FIG. 7G, Metabolic activity per unit of DNA reveals no differences in metabolic activity of MSCs. In FIG. 7H, Rhodamine-Phalloidin/DAPI staining shows consistency in MSC number and phenotypes in ECM from all kidney regions. Scale bars: 50 μm.

KSCs and MSCs were seeded at equal densities on decellularized kidney ECM sheets (FIGS. 7A, 7E), cultured for 48 hrs, assayed for DNA and metabolic activity, and data were normalized to those measured for cells grown on tissue culture plastic.

DNA quantification of KSCs cultured on decellularized ECM sheets revealed significant differences between cortex, medulla, and papilla regions, with papilla ECM again yielding the fewest KSCs (FIG. 7B). Metabolic activity per unit DNA confirms that KSCs on papilla were significantly more metabolically active per cell than were KSCs on either cortex or medulla (FIG. 7C). No significant differences in metabolism between KSCs on cortex and medulla ECM sheets were observed.

In addition to differences in cell number, distinct morphologies and orientation of KSCs were observed in cortex, medulla, and papilla ECM (FIG. 7D). No significant differences in DNA, metabolic activity, or morphology were observed for MSCs on kidney region ECM sheets (FIGS. 7F, 7G, 7H). Significantly more KSCs were observed on decellularized kidney region ECM when compared to MSCs after 48 hrs (FIGS. 7B, 7F).

Structure Formation by KSCs on Kidney ECM Sheets

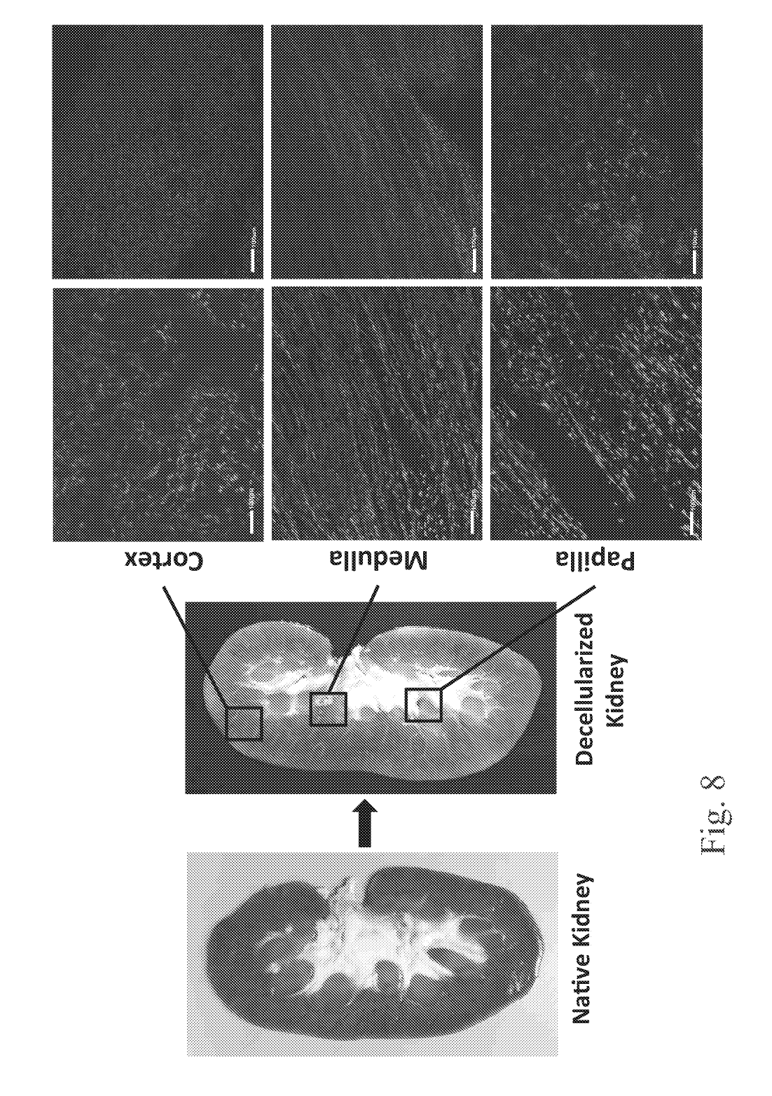

Referring to FIG. 8, Live/Dead and rhodamine/phalloidin staining of KSCs and MSCs on regionally specific decellularized kidney region ECM sheets are illustrated. KSCs cultured decellularized kidney ECM from different regions display significantly different cell number, morphology, orientation, and structure formation at 48 hours. KSCs on cortex sheets show star-like morphology in random aggregations, whereas KSCs on medulla exhibit elongated morphology with significant aligning and formation of tubular structures. KSCs on papilla show some aligning but also periodic rounded morphology not seen in cortex or medulla. Rhodamine-phalloidin staining highlights significant differences in KSC morphology and orientation between kidney regions after 7 days. The corresponding cultures of MSCs show no differences cell number, morphology, or alignment. Scale bars: 100 μm.

KSCs were seeded at equal densities onto ECM sheets derived from decellularized kidney regions (cortex, medulla, and papilla), cultured for 48 hrs or 7 days, and imaged. KSCs showed clear differences when cultured on ECM sheets from different kidney regions in cell morphology, orientation, and structure formation already by 48 hrs of cultivation (FIG. 8). On decellularized cortex sheets, KSCs consistently showed star-like morphology and regional aggregations (FIG. 8, top). On decellularized medulla, KSCs displayed elongated morphology, end-to-end alignment, and tubular formations distinctly not seen in decellularized cortex at 48 hrs (FIG. 8, middle). On decellularized papilla, KSCs appeared morphologically different from KSCs on cortex, with some alignment in the upper papilla similar to KSCs on medulla (FIG. 8, bottom). Additionally, KSCs with a rounded morphology were periodically observed in decellularized papilla sheets, only rarely in medulla, and not in cortex. KSCs on cortex displayed structures resembling renal corpuscles similar to those seen in native cortex H&E histological sections, while KSCs on medulla displayed many straight tubular bundles similar to the medullary rays seen in medulla H&E histological sections.

Metabolic Activity of KSCs on Whole Organ ECM

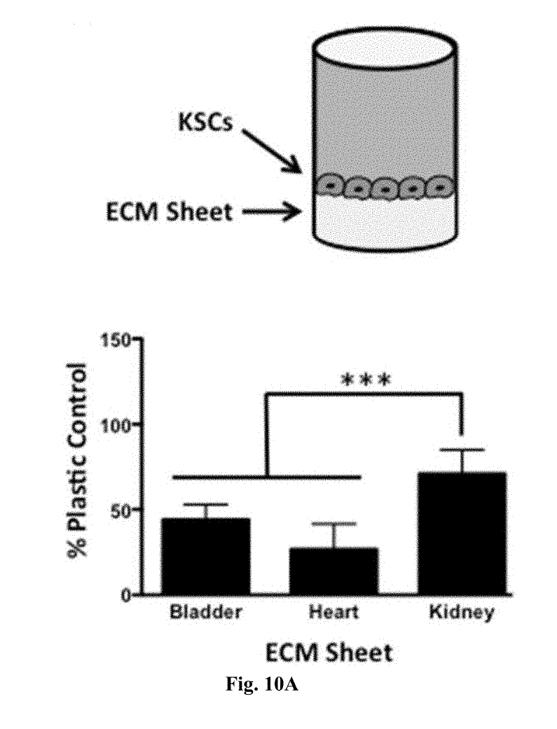

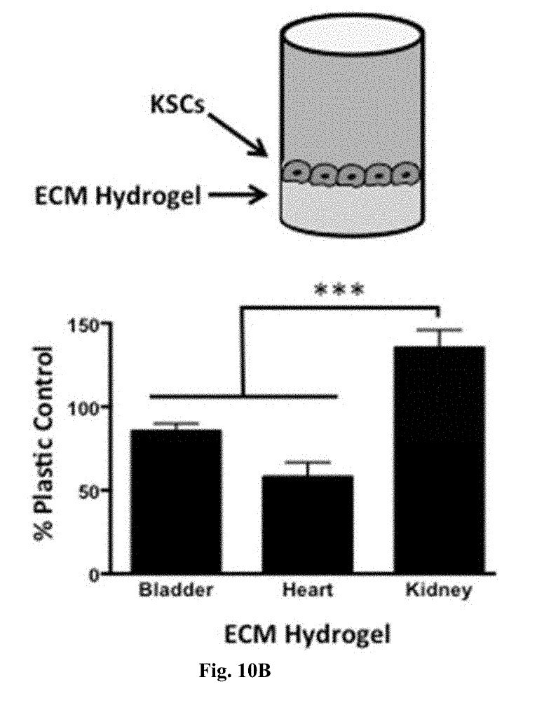

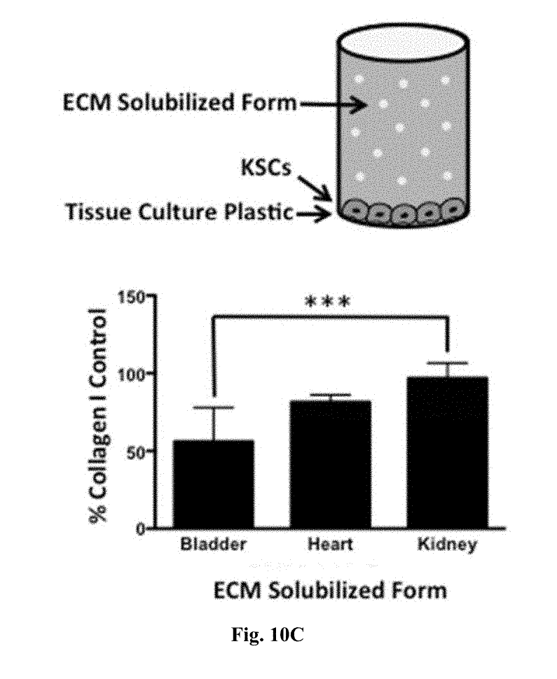

Kidney stem cells (KSCs) were seeded onto tissue culture plastic and cultured for 48 hrs in three different forms of ECM (decellularized sheets, hydrogels, and solubilized forms) obtained from porcine hearts, bladders and kidneys. KSCs grown on decellularized whole kidney sheets and whole kidney hydrogel showed significantly higher metabolic activity at 48 hrs when compared to KSCs grown on bladder and heart ECM sheets and hydrogels (FIGS. 10A, 10B). Furthermore, KSCs cultured on tissue culture plastic in the presence of solubilized whole kidney ECM were more metabolically active than KSCs cultured in the presence of solubilized bladder and heart ECM, with a significant difference between cells cultured in the presence of kidney and bladder ECM (FIG. 10C). These results indicate that KSCs cultured on or in the presence of whole kidney ECM in various forms—decellularized sheets, hydrogels, and solubilized forms—are significantly more metabolically active than KSCs cultured in various forms of ECM from other organs, suggesting a degree of recognition or specificity by endogenous kidney stem cells to the ECM of their native organ.

Chemotaxis (Transwell) Assay

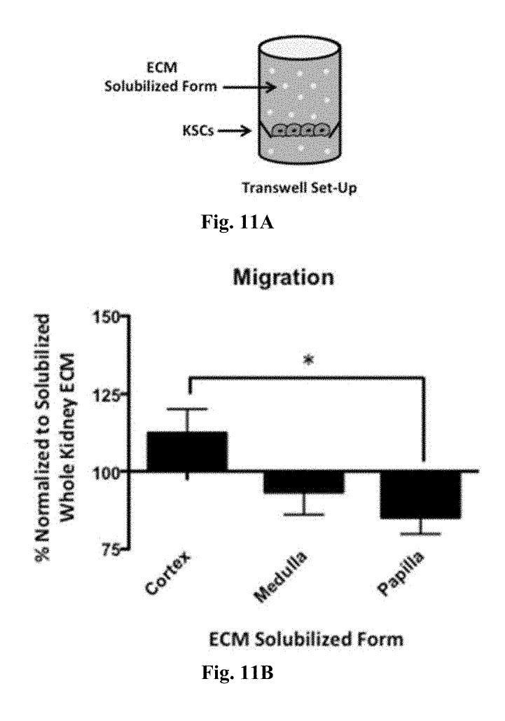

KSCs seeded onto transwells with 8 μm pores were cultured in the presence of solubilized kidney region ECM (FIG. 11A). KSCs cultured in the presence of solubilized papilla ECM demonstrated the least chemotaxis across the membrane, while KSCs cultured in solubilized cortex ECM the most chemotaxis (FIG. 11B).

### DISCUSSION

The present disclosure provides ECM biomaterials in various formats including acellular scaffolds, sponges, hydrogels, and solutions. These materials are derived from various tissues such as adrenal gland, bladder, blood vessel, brain, breast, bone, esophagus, heart, kidney, larynx, liver, lung, lymph node, muscle, parathyroid, pancreas, placenta, skin, small intestine, spleen, stomach, testes, thymus, thyroid, umbilical cord, and uterus. Tissues may be from various sources such as human and animal; fetal, juvenile, and adult; healthy, diseased, and transgenic. These ECM biomaterials modulate stem cells in a region-specific manner. For example, data show that there is a significant degree of recognition and specificity between adult kidney stem cells and their extracellular environment. KSCs showed significantly higher proliferation and higher metabolic activity in kidney ECM when compared to KSCs in ECM from other organs (FIG. 9). In addition, KSCs showed lower proliferation and higher metabolic activity when cultured in papilla ECM (kidney stem cell niche) compared to medulla and cortex ECM. The decrease in cell proliferation by ECM is of great interest since in vivo the kidney papilla shows little cycling activity. These effects were not observed with bone marrow-derived MSCs cultured under the same conditions, and the observed differences were independent of the form of the EMC, i.e., sheet vs. hydrogel vs. solubilized form (FIGS. 5, 6, 7).

Kidney stem cells cultured on whole kidney ECM were compared to ECM derived from the urinary bladder and heart to determine if there was recognition between KSCs and the ECM at the organ level. ECM from whole bladder, heart, and kidney was prepared in three different forms: decellularized sheets, hydrogels, and solubilized forms. KSCs were significantly more proliferative and metabolically active in all three forms of kidney ECM when compared to respective forms of bladder or heart ECM (FIG. 9).

The organ specificity of KSCs according to the present disclosure demonstrates specificity of liver sinusoidal endothelial cells to liver ECM and indicates that decellularized kidney ECM sheets contain organ-specific cues. Higher KSC metabolism is observed in whole kidney ECM hydrogel and soluble ECM, where the ECM ultra-structure is absent and only a homogenous mix of digested ECM proteins (cross-linked in the hydrogel or dissolved in solution) comprises the extracellular environment (FIGS. 9B, 9C). Taken together, these data indicate that the interactions responsible for cell-matrix recognition is not limited to structural cues from decellularized matrix but also relies on signaling from small molecules or protein fragments.

A degree of kidney stem cell-matrix specificity has been shown at the organ level. Accordingly methods are provided to isolate and prepare ECM biomaterials from three distinct regions of the kidney—the cortex, medulla, and papilla—to show cell-matrix interactions at the regional level. Each region of the kidney harbors a variety of cell types and structures, including extensive networks of tubules, collecting ducts, and capillaries, necessary for filtering blood or concentrating urine. In an adult mouse kidney, label-retaining cells (KSCs) remain quiescent in the renal papilla (stem cell niche) and migrate to the site of injury following renal ischemia. Consequently, this adult kidney stem cell population may be used to investigate region-specific effects of kidney ECM on the proliferation and metabolism of KSCs.

Characterization of the ECM in native cortex, medulla, and papilla reveals significant differences in structure and composition, many of which are retained after decellularization and further processing. Following the removal of >99% nuclear material (FIGS. 2A, 2D), some ECM proteins—such as collagens I and IV, were preserved similarly across regions of the kidney (FIGS. 2B, 2E, 4A), while the amount of sGAG (FIGS. 2C, 2F) as well as overall ECM ultra-structure (FIG. 3) differed significantly across regions. In all regions, the cell adhesion molecule fibronectin was significantly depleted after decellularization (FIG. 4B).

Scanning electron micrographs of kidney region ECM showed comparable topographies between native and decellularized sections, indicating that many ultra-structural features of the ECM are retained after cells are removed. Additionally, large tubular collecting ducts approximately 50 μm in diameter are seen in decellularized papilla sections (FIG. 3B) and demonstrate distinguishing ultra-structural features of the KSC niche not found in medulla or cortex. Given the sensitivity of stem cells to their environment, such differences in matrix architecture between kidney regions may account for differences observed in KSC activity and morphology.

While structural cues account for some organ or even region-specific signaling to kidney stem cells, compositional cues from the ECM also play a role in informing KSCs about their extracellular environment. Decellularized whole mouse kidney ECM are able to direct the differentiation of embryonic stem cells into specialized cells types as well as to encourage proliferation along the basement membrane, indicating that the basement membrane or one or more of its components promotes signaling for proliferation. Differences in the composition and distribution of the basement membrane in different regions of the kidney thus account for some of the region-specific differences observed in KSC proliferation and metabolism. Further, kidney region ECM biomaterials may be used to selectively differentiate KSCs into region-specific cell types.

As shown in FIG. 7D, KSCs showed significant differences in cell number, morphology, and arrangement as a function of the region from which the ECM was derived. Such differences are not observed with MSCs (FIG. 7H), indicating that structural and/or compositional differences in the kidney ECM are recognized by KSCs. In FIG. 8, KSCs seeded on the cortex show arrangement into distinct circular shapes similar to the renal corpuscular structures seen in the immunostaining of native cortex sections (FIG. 4A). Together these data indicate that the KSCs were able to recognize the type of matrix and adopt morphology similar to the native tissue. Further, KSCs may be differentiating into one or multiple cells types in situ.

Across the ECM regions discussed above, KSCs cultured in papilla ECM consistently showed significantly lower cell number (DNA content) when compared to KSCs in cortex and medulla ECM (FIGS. 5B, 6B, 7B). Similar trends in mitochondrial activity and cell number are observed at two different time points (48 hours and 7 days) when cultured in three different forms of kidney ECM: sheets, hydrogels, and solubilized forms (FIGS. 5C, 6C, 7C). This trend indicates that the effect of the ECM on KSCs' growth and metabolic activity does not depend on the structural form of the ECM but rather on the composition. This is further supported by the trend in metabolic activity when cultured in hydrated or solubilized forms of the ECM where high metabolic activity was found in the papilla ECM and low metabolic activity in the cortex and medulla ECM.

When cultured in ECM obtained from entire kidney sections (containing cortex, medulla, and papilla), the metabolic activity was found to be within the values obtained for the individual regions, suggesting a dose effect. In addition, factors may be more readily available in the solubilized form but may still be locked into place or obscured by other proteins in an intact decellularized sheet.

One aspect of this work is the development of tissue-specific biomaterials and the potential for tissue regeneration using regionalized ECM biomaterials to direct the differentiation of reparative stem cells, for example to address renal pathologies such as diabetes or kidney failure. This approach translates into other regionalized organs as well. Cultivation of epithelial and endothelial cells on fully decellularized rat kidney scaffolds in a whole-organ perfused bioreactor results in a bioengineered kidney that produced rudimentary urine in vitro (in the bioreactor) and in vivo (following orthotopic implantation in rat). A variety of cell and tissue engineering applications may be applied in conjunction with regionalized ECMs. For example, since the renal papilla is the KSC niche, it may be used to maintain the cells in a stem-like state in vitro, while cortex and medulla ECM may be used to differentiate KSCs into other renal cell types.

Ischemic conditions in the cortex encourage mobilization, migration, and differentiation of quiescent KSCs in the papilla. The data in FIG. 11B support these findings. Solubilized factors from damaged matrix could provide chemotactic cues for KSCs, signaling them to migrate to the site of injury and differentiate into cell types for repair and regeneration. In addition, ECM hydrogels allow for production in large quantities when compared to ECM sheets (by pooling ECM from a large number of kidneys) as well as the use of ECM derived from regions that are small in size or volume, such as the renal papilla.

With regard to FIG. 9, Organ-Specific Effects of ECM on Metabolism of Kidney Stem Cells are illustrated. In FIG. 9A, Kidney stem cells (KSCs) show significantly higher metabolic activity when cultured on decellularized ECM sheets derived from kidney as compared to either bladder or heart ECM sheets. Data are normalized to cells grown on tissue culture (TC) plastic. In FIG. 9B, KSCs cultured on whole bladder, heart, and kidney ECM hydrogels also showed significantly higher metabolic activity for kidney ECM than other organ hydrogels when normalized to cells grown on TC plastic. Data are normalized to cells grown on tissue culture (TC) plastic. In FIG. 9C, After 24-hr starvation, KSCs cultured on TC plastic showed higher metabolic activity in media supplemented with solubilized (digested) whole kidney ECM than solubilized ECM from other organs.

With regard to FIG. 10, Characterization of Solubilized Kidney Region ECM and ECM Hydrogels is illustrated. In FIG. 10A, Gel electrophoresis of solubilized kidney ECM digests and collagen I. In FIG. 10B, Collagen and sGAG quantification of solubilized kidney ECM. C Turbidimetric gelation kinetics of kidney region hydrogels. In FIG. 10D, Kidney region ECM hydrogels.

With regard to FIG. 11, Chemotaxis of KSCs in the Presence of Solubilized Kidney Region ECM. In FIG. 11A, Transwell chemotaxis assay experimental set-up is illustrated. KSCs are seeded on an 8 mm porous membrane and allowed to migrate through the membrane in the presence of different solubilized ECM chemotactic factors. In FIG. 11B, KSCs in solubilized papilla ECM showed the least amount of chemotaxis relative to KSCs cultured in solubilized cortex and medulla ECM. Solubilized cortex ECM instigated significantly more KSC chemotaxis than solubilized papilla ECM.

ECM biomaterials derived from the tissues affect the growth and metabolism of stem cells with regional specificity. Region-specific ECM may thus provide an optimal substrate for the in vitro cultivation or the delivery of therapeutic stem cells and their derivatives. The present disclosure is application to development of biomaterials or applications in tissue repair and regeneration using regionalized ECM biomaterials to deliver and direct the differentiation of reparative stem cells to address pathologies such as diabetes or kidney failure.

### EXAMPLES

Decellularization

Porcine bladders, hearts, and kidneys were procured from Yorkshire pigs (65-70 kg) immediately following euthanasia, excess tissue was trimmed, and the blood and debris removed with water. The organs were stored at −80° C. for at least 24 hrs, thawed and then sliced into <2 mm thin cross-sections. Cross-sections from the middle third of the kidney were separated into cortical, medullary, and papillary regions. Whole kidneys and kidney regions were decellularized using a modification of a previously established method. Briefly, the slices were washed with 2× phosphate-buffered saline (PBS) for 15 min, followed by 2 hrs of 0.02% trypsin, 2 hrs of 3% Tween-20, and 2 hrs of 4% sodium deoxycholate treatment. After each step, kidney sections were washed with 2×PBS for 15 min. Kidney ECM slices were treated for 1 hr with 0.1% peracetic acid and subjected to alternating sterile 1×PBS and dH₂O washes.

Histological Analysis

Native and decellularized tissue samples were fixed in formalin, embedded in paraffin, sectioned at 5 μm thickness, stained with hematoxylin and eosin, Trichrome, or Alcian Blue, and imaged using an Olympus IX81 microscope at 10×.

DNA Quantification of Decellularized Tissue

DNA content of decellularized tissue was quantified using Quanti-iT PicoGreen dsDNA Assay kit (Invitrogen) according to the manufacturer's instructions. Briefly, tissue samples were weighed, digested overnight with Proteinase K in TEX buffer at 56° C., and mixed with PicoGreen reagent. Fluorescence emission was measured at 520 nm with excitation at 480 nm, and DNA was quantified using a standard curve.

ECM Characterization

Collagen content of kidney region sheets/digests was determined using the Sircol collagen assay kit (Biocolor). Samples were digested in 0.1 mg pepsin/mL overnight at room temperature (25° C.), and the Sircol assay was performed according to the manufacturer's instructions. Sulfated glycosaminoglycan (sGAG) content of kidney region sheets/digests was determined using the 1, 9-dimethylene blue (DMB) dye binding assay. Samples were digested in 125 μg papain/mL overnight at 60° C. sGAG content was quantified by mixing ECM digest samples with DMB dye in a 1:5 ratio and reading spectrophotometric absorbance at 595 nm and 540 nm. The difference in absorbance at these wavelengths was used with a chondroitin-6-sulphate standard curve to quantify sGAG content. Pepsin digests of the regional kidney ECM and collagen I (BD, Biosciences) were electrophoresed on 7.5% polyacrylamide gels (BioRad) under reducing conditions (5% 2-mercaptoethanol). The proteins were visualized with Coomassie Brilliant Blue (BioRad) and imaged by scanning the polyacrylamide gel.

Scanning Electron Microscopy (SEM)

Native and decellularized sections of regional kidney ECM were fixed in formalin, rinsed in 70% EtOH, frozen, lyophilized, and gold-coated (5 nm thickness). Sections were imaged on a Hitachi S-4700 FE-SEM with accelerating voltage 2.5 kV.

Immunohistochemical Staining

Sections of native and decellularized kidney ECM were fixed in formalin for 30 min, embedded in paraffin, cut to 5 μm, and mounted on slides. Sections were deparaffinized and subjected to boiling citrate buffer (pH=6.0) for 16 minutes for antigen retrieval, and blocked with 10% Normal Goat Serum in PBS for 2 hrs at room temperature. Primary antibody staining was performed for 2 hrs at 4° C. using the following primary antibodies and dilutions: Collagen IV (Rb pAb to Coll IV, ab6586) diluted 1:200 and Fibronectin (Rb pAb to Fibronectin (ab23750)) diluted 1:200. For all stains, the secondary antibody (Goat pAb to Rb IgG (ab98464)) was diluted 1:200 and incubated for 1 hr at room temperature. Sections were mounted in Vectashield Mounting Medium with DAPI, cover slipped, and imaged with an Olympus IX81 microscope at 10×.

Mouse Kidney Stem Cells

Mouse kidney stem cells (KSCs) were obtained from mouse kidneys as previously described, cultured in Dulbecco's Modified Eagle Medium (DMEM) with high glucose supplemented with 10% fetal bovine serum (FBS) and 1% penicillin/streptomycin under standard culture conditions (37° C. and 5% CO₂).

Mouse Mesenchymal Stem Cells

Mouse mesenchymal stem cells (MSCs) were obtained from Texas A&M Health Science Center College of Medicine Institute for Regenerative Medicine and cultured in Iscove's Modified Dulbecco's Medium (IMDM) supplemented with 10% FBS, 10% horse serum, and 1% penicillin/streptomycin under standard culture conditions (37° C. and 5% CO₂).

Preparation of ECM Sheets, ECM Hydrogels, and Solubilized ECM

Decellularized whole organ and regional kidney slices were either perforated with a 7 mm biopsy punch into sheets or snap-frozen in liquid nitrogen. Sheets were stored in 1X PBS at 4° C. until use while frozen pieces were pulverized into a fine powder using a mortar and pestle, and lyophilized for 24 hrs. Lyophilized ECM powder was digested as previously described. Briefly, 1 g of lyophilized ECM powder was mixed with 0.1 g pepsin (Sigma, ˜2500 U/mg) in 0.01M HCl. The solution was allowed to digest for 48 hrs at room temperature (25° C.) under constant stirring. Final digests were aliquotted and stored at −80° C. until use. The soluble ECM was obtained by neutralizing ECM stock digests and added to cell culture media directly (Typically 1 mg dry ECM/ml medium). Hydrogels were prepared as previously described by mixing ECM stock digests with 1×PBS, 10×PBS, and 0.1M NaOH to yield a hydrogel with a final concentration of 6 mg/mL at 4° C.

Solubilized Mitogenicity Assay

KSCs and MSCs were seeded on tissue culture plastic (TCP) at 2.5×10⁴ cells/mL, cultured for 24 hrs in media supplemented with 10% FBS, and starved for 24 hrs in media containing 0.5% FBS. Next, ECM digests were added to the media (0.1 mg/mL) with 0.5% FBS for 48 hrs. On the fourth day, culture media was replaced with media containing 10% Alamar Blue® (Invitrogen) and the cells were incubated for 14 hrs. Culture media were transferred into new 96-well plates and absorbance was measured at 570 nm and normalized to 600 nm.

DNA Quantification of Seeded ECM Sheets and Hydrogels

DNA content of seeded ECM sheets and hydrogels was quantified using Quanti-iT PicoGreen dsDNA Assay kit (Invitrogen) according to the manufacturer's instructions. After 48 hrs of culture, samples were digested in 125 μg papain/mL overnight at 60° C. and mixed with PicoGreen reagent. Fluorescence emission was measured at 520 nm with excitation at 480 nm, and DNA was quantified using a standard curve.

Metabolic Activity

ECM sheets were glued to the bottom of 96-well plates using 2% fibrin. ECM and collagen I hydrogels were prepared in 96-well plates by adding 50 μL of hydrogel (neutralized and brought to the concentration of 6 mg/ml) at 4° C. The plates containing the hydrogels were incubated for 40 minutes at 37° C. until gelation was observed. KSCs and MSCs were grown under standard culture conditions, trypsinized, seeded into the ECM sheets or hydrogels at 2.5×10⁴ cells/mL, and cultured for 48 hr or 7 days. After a 48-hr incubation, the culture media was replaced with media containing 10% Alamar Blue® (Invitrogen). After 14-hr incubation, media were transferred into new 96-well plates and absorbance was measured at 570 nm and normalized to 600 nm.

Confocal Imaging

KSCs and MSCs grown under standard culture conditions were seeded into ECM sheets or hydrogels at 2.5×10⁴ cells/mL and cultured for 48 hrs or 7 days, at which times they were stained with Live/Dead Viability Kit (Invitrogen) or fixed with 3.7% formaldehyde and stained with rhodamine phalloidin (Invitrogen) and DAPI according to the manufacturer's instructions. Confocal imaging was performed using an Olympus Fluoview FV1000 Confocal Microscope.

Gelation Kinetics

Regional kidney ECM hydrogels and collagen I hydrogels were prepared as previously described. Gelation kinetics were determined spectrophotometrically as previously described. Briefly, gel solutions at 4° C. were transferred to a cold 96-well plate by adding 100 μL/well in triplicate. The SpectraMax spectrophotometer was pre-heated to 37° C., plate was loaded, and turbidity measured at 405 nm every 2 min for 1.5 hrs. Absorbance values were recorded for each well and averaged. Three separate tests were performed on two separate batches of kidney ECM hydrogels.

Chemotaxis (Transwell) Assay

KSCs were cultured for 24 hrs in 0.5% FBS, trypsinized, and seeded onto transwells with 8 μm pores. Region solubilized kidney ECM was added to the media at a concentration of 0.1 mg/mL. After 6 hrs, transwells were collected, attached cells removed from the top of the membrane using a Q-tip, and membranes were detached. DNA from cells attached to the bottoms of the detached membranes was quantified with CyQuant® Direct Cell Proliferation Assay Kit according to the manufacturer's instructions. Fluorescence emission was measured at 535 nm with excitation at 480 nm, and DNA was quantified using a standard curve.

Statistical Analysis

One-way ANOVA test with Tukey's multiple comparison post hoc test and two-way ANOVA test with Bonferroni post hoc test were performed using Prism v6 (GraphPad, La Jolla Calif.). A p<0.05 was considered statistically significant.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A culture medium comprising:
a scaffold, the scaffold comprising decellularized extracellular matrix.

2. The culture medium of claim 1, wherein the scaffold comprises a substantially planar sheet.

3. The culture medium of claim 1, wherein the decellularized extracellular matrix is selected from the group consisting of: adrenal gland extracellular matrix, bladder extracellular matrix, blood vessel extracellular matrix, brain extracellular matrix, breast extracellular matrix, bone extracellular matrix, esophagus extracellular matrix, heart extracellular matrix, kidney extracellular matrix, larynx extracellular matrix, liver extracellular matrix, lung extracellular matrix, lymph node extracellular matrix, muscle extracellular matrix, parathyroid extracellular matrix, pancreas extracellular matrix, placenta extracellular matrix, skin extracellular matrix, small intestine extracellular matrix, spleen extracellular matrix, stomach extracellular matrix, testes extracellular matrix, thymus extracellular matrix, thyroid extracellular matrix, umbilical cord extracellular matrix, and uterus extracellular matrix.

4. The culture medium of claim 1, wherein the decellularized extracellular matrix is a region-specific extracellular matrix.

5. The culture medium of claim 1, wherein the decellularized extracellular matrix is an organ-specific extracellular matrix.

6. The culture medium of claim 1, wherein the decellularized extracellular matrix is extracellular matrix of a region of an organ.

7. The culture medium of claim 6, wherein the organ is the kidney and the region is selected from the group consisting of: cortex, medulla, and papilla.

8. A kit for making a culture medium, the kit comprising:
a solution comprising decellularized extracellular matrix;
at least one reagent adapted to reconstitute the solution into a hydrogel.

9. The kit of claim 8, wherein the reagent comprises phosphate buffered saline or sodium hydroxide.

10. The kit of claim 8, wherein the decellularized extracellular matrix is selected from the group consisting of: adrenal gland extracellular matrix, bladder extracellular matrix, blood vessel extracellular matrix, brain extracellular matrix, breast extracellular matrix, bone extracellular matrix, esophagus extracellular matrix, heart extracellular matrix, kidney extracellular matrix, larynx extracellular matrix, liver extracellular matrix, lung extracellular matrix, lymph node extracellular matrix, muscle extracellular matrix, parathyroid extracellular matrix, pancreas extracellular matrix, placenta extracellular matrix, skin extracellular matrix, small intestine extracellular matrix, spleen extracellular matrix, stomach extracellular matrix, testes extracellular matrix, thymus extracellular matrix, thyroid extracellular matrix, umbilical cord extracellular matrix, and uterus extracellular matrix.

11. The kit of claim 8, wherein the decellularized extracellular matrix is a region-specific extracellular matrix.

12. The kit of claim 8, wherein the decellularized extracellular matrix is an organ-specific extracellular matrix.

13. The kit of claim 8, wherein the decellularized extracellular matrix is extracellular matrix of a region of an organ.

14. The kit of claim 8, wherein the organ is the kidney and the region is selected from the group consisting of: cortex, medulla, and papilla.

15. A culture medium comprising:
a hydrogel, the hydrogel comprising decellularized extracellular matrix.

16. A culture medium comprising:
solubilized decellularized extracellular matrix.

17. A culture medium comprising:
a sponge, the sponge comprising decellularized extracellular matrix.

18. A method comprising:
extracting a portion of an organ;
decellularizing the portion of the organ to yield extracellular matrix;
powdering the extracellular matrix to yield a powder;
digesting the powder to yield a digest; and
reconstituting the digest into a hydrogel.

19. The method of claim 18, wherein the organ is a kidney and the portion is extracted from one of the group consisting of: cortex, medulla, and papilla.

20. The method of claim 18, wherein the organ is selected from the group consisting of: adrenal gland, bladder, blood vessel, brain, breast, bone, esophagus, heart, kidney, larynx, liver, lung, lymph node, muscle, parathyroid, pancreas, placenta, skin, small intestine, spleen, stomach, testes, thymus, thyroid, umbilical cord, and uterus.

21. The method of claim 18, wherein decellularizing the portion of the organ comprises at least one of:
treating with trypsin;
treating with Tween-20;
treating with sodium deoxycholate; and
treating with peracetic acid.

22. The method of claim 18, wherein powdering the extracellular matrix comprises:
snap freezing the extracellular matrix;
pulverizing the extracellular matrix; and
lyophilizing the extracellular matrix.

23. The method of claim 18, wherein digesting the powder comprises:
treating with pepsin; and
treating with hydrochloric acid.

24. The method of claim 18, wherein reconstituting the hydrogel comprises:
introducing pepsin; and
introducing hydrochloric acid.

25. The method of claim 18, further comprising:
culturing stem cells in the hydrogel.

26. The method of claim 18, further comprising:
introducing the hydrogel into an organ in vivo.

27. A method comprising:
extracting a portion of an organ;
decellularizing the portion of the organ to yield extracellular matrix;
powdering the extracellular matrix to yield a powder;
digesting the powder to yield a digest; and
centrifuging, vortexing, and lyophilizing the digest to yield a sponge.
